(19) United States
(12) Patent Application Publication
(10) Pub. No.: US 2010/0303803 A1
(43) Pub. Date: Dec. 2, 2010

(54) CATENATE FOR IMMUNOSTIMULATION

(75) Inventors: Matthias Schroff, Berlin (DE); Burghardt Wittig, Berlin (DE); Manuel Schmidt, Berlin (DE); Janine Löhr, Berlin (DE); Christiane Kleuss, Berlin (DE)

Correspondence Address: URSULA B. DAY, ESQ., 708 Third Avenue, SUITE 1501, NEW YORK, NY, 10017, US

(73) Assignee: Mologen AG, Berlin (DE)

(21) Appl. No.: 12/742,000

(22) PCT Filed: Nov. 7, 2008
(86) PCT No.: PCT/EP08/09618

§ 371 Date: Jul. 14, 2010

(30) Foreign Application Priority Data
Nov. 7, 2007 (EP) 07075967.5

(51) Int. Cl. A61K 39/395; C07H 21/02; A61K 31/337; A61K 38/46; A61K 31/17; A61K 38/12; A61K 31/662; A61K 31/196; A61K 31/255; A61K 31/175; A61K 31/7008; A61K 31/166; A61K 31/675; A61K 33/24; A61K 31/282; A61K 31/704; A61K 31/4375; A61P 35/00

(52) U.S. Cl. 424/130.1; 536/23.1; 514/449; 424/94.6; 514/588; 514/21.1; 514/110; 514/564; 514/567; 514/517; 514/589; 514/275; 514/23; 514/615; 514/83; 424/649; 514/492; 514/34; 514/282

(57) ABSTRACT

The invention relates to a multimeric, non-coding nucleic acid molecule for modulating the activity of the human or animal immune system, and to a production method therefor, and to a vaccine containing said multimeric, non-coding nucleic acid molecule. Said multimeric, non-coding nucleic acid molecules can be non-coding nucleic acid molecules that consist of at least two of said molecules (dimer) or assemblies of several non-coding nucleic acid molecules.

The present invention relates to a multimeric, non-coding nucleic acid molecule for modulation of the activity of the human and animal immune system as well as a method for the manufacture thereof and a vaccine, comprising the multimeric, non-coding nucleic acid molecule, wherein multimeric, non-coding nucleic acid molecules may be understood as non-coding nucleic acid molecules, comprising at least two catenated molecules (dimer) of said non-coding nucleic acid molecules.

As the adaptive immune response starts with a delay (3-5 days) after selection of the specific lymphocytes for the respective pathogen and their clonal expansion and differentiation and then provides a long lasting protection for the respective pathogen by forming an immunological memory, the cells of the innate immune system recognize pathogens via conserved pathogen associated molecular patterns (PAMP) by germ cell encoded receptors and react immediately. Different reactions belong to different kinds of cell types like the secretion of cytokines (e.g. IL-1, IL-6, TNF-α) and chemokines (e.g. IL-8/CXCL8, MIP-1α/β, MCP-1), the activation of effectors mechanisms (phagocytosis, respiratory discharge, liberation of bactericide substances or lytic granules), the expression of co-stimulatory molecules (CD80, CD86) as well as the enhanced expression of MHC-molecules. Thereby on one hand effector cells are recruited and activated, which are able to eliminate the entered pathogen and on the other hand the cells of the adaptive immune system receive the necessary signals for their activation.

In order to improve the immune response CpG-oligonucleotides (CpC-ODN) have been used as a new class of immune modulating molecules. Such non-methylated CG-motives can be found in bacterial DNA and represent a “danger signal” for the immune system. As pathogen associated molecular pattern (PAMP) they cause the unspecific activation of the innate immune system (Krieg, Nat. Med. 2003, 9: 831-835). CpG-ODN induce via the cytokines interleukine-12, interferon-χ and tumor necrosis factor-a a T_{H}1-based immune response.

Immune stimulatory nucleic acid sequences (ISS), comprising said CpG-ODN, have a length of several bases and comprise no open reading frame for the expression of proteins.

The ISS represent linear nucleic acid molecules, which ends are open (free hydroxyl- and phosphate groups) or protected by synthetic groups.

The strong stimulation of the cellular immune response allows influencing the feedback loops, which will not result in a satisfactory immune activity for the patient without intervention.

The modification of CpC-ODN with a phosphothioate-backbone, which is used for stabilizing the CpG-DNA, has several severe disadvantages. The noted toxicity belongs especially to this (Heikenwalder 2004, Levin 1999) as well as unspecific binding to proteins (Brown 1994).

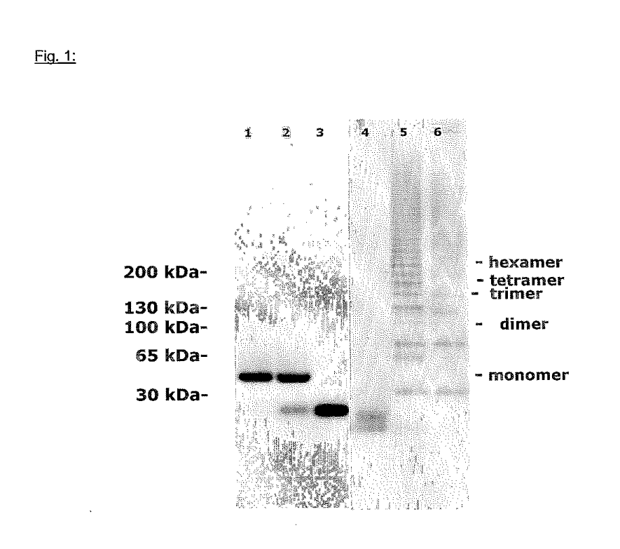

Due to this a new class of covalently closed immune stimulatory DNA was developed (WO 01/07055/EP 1196178). These DNA-molecules consist of two chemically synthesized DNA-molecules, with a self complementary part at the 5′- and at the 3′-end with palindromic, overlapping ends, so that ligation of both DNA-molecules results in a covalently closed molecule. These DNA-molecules with CG-motives in the non-complementary part show a similar activity as CpG-ODN (enhanced expression of the surface molecules CD80, CD40, MHC on B-cells and secretion of IL-6, IFN-γ, IFN-a IL-12, TNFα by PBMC), but they show in comparison to CpG-ODN with phosphorothioate backbone differences with regard to the expression pattern of the induced cytokines and a clearly lesser toxicity in mice. This immune stimulatory DNA from the state of the art has with regard to the modulation of the activity of the human and animal immune system several disadvantages. It is not possible, to modulate the activity of the human and animal immune system in a desired degree, especially to activate. The molecules according to WO 01/07055, as shown for example in FIG. 1 or in claim 11, consist of several deoxyribonucleotide rests which form a partly single stranded dumbbell-shaped and covalently closed DNA molecule, which is designated within the sense of the present invention as a monomer. According to the WO 01/07055 these monomers made from oligonucleotides have been heated before ligation, receiving uniform molecules out of the oligonucleotides, each consisting of a dumbbell-shaped monomer (compare FIG. 1 of the WO 01/07055). It is known to a person skilled in the art, how to interpret the term monomer consisting of oligonucleotides. The artisan knows that monomers may consist out of several molecular elements like oligonucleotide rests, without loosing their character as monomer (e.g. myoglobin is with 153 amino acids a monomer). The monomer is a dumbbell according to FIG. 1 of WO 01/07055. Monomers in the sense of the invention do not designate a structure consisting for instance out of a single base, but does designate a closed dumbbell-shaped form, consisting of nucleotides, which consist their self out of several deoxyribonucleotide rests (compare FIG. 1 or claim 11 of WO 01/07055).

Coming from this state of the art it is an objective of the present invention to provide suitable immune stimulatory DNA molecules, which initiate an improved immune response, as well as method for their manufacture as well as vaccines, comprising said immune stimulatory DNA-molecules.

Immune stimulation means in the context of the present invention that the mediator and effector cells of the immune system, thus mainly the presently known thymocytes with helper function and the cytotoxic thymocytes, B-cells and so called NK (natural killer)-cells, macrophages and monocytes as well as dendritic cells and their precursors, as well as cell populations with so far not clearly identified functions within the immune system, are stimulated by the use of nucleic acid molecules for proliferation, migration, differentiation or their activity. Immune modulation means, that besides a general stimulation in the above defined sense also the type or character of the immune reaction will be influenced, whether by affecting a beginning or maturing immune reaction or by changing an established reaction with regard to their character.

The present invention solves the objective by providing an oligo-respectively multimeric, non-coding nucleic acid molecule. It was completely surprising that dimers, trimers, pentamers or mulitmers of eovalently closed immune stimulatory DNA has an surprisingly improved effect with regard to the molecules known from the state of the art. The invented multimeric molecule can be manufactured by a method, comprising the following steps:
providing a 5′-phosphorylated oligonucleotide,
alcohol precipitation or lyophilisation, especially in the presence of MgCl₂, until a dry residue is obtained, followed by resuspension in a buffer.
adding T4-DNA-ligase, thereby producing a reaction mixture, and
incubation of the reaction mixture at 37° C. for at least 30 minutes.

A molecule according to the invention can be characterized by its method of manufacture. The method of manufacture serves as definition for the product. The product defined by the method is new with regard to molecules described in the state of the art, like for example in the WO 01/07055. The molecule, which is described and claimed by its way of manufacture, is defined by its structural and functional properties, which result from the application of the method of its manufacture for a person skilled in the art. The manufacture of the molecule is very precise using the way of manufacture, because the characterization by structural features is not feasible. The claimed method can be performed successfully, because all necessary declarations for a person skilled in the art are disclosed. The method for manufacture further differentiates from known methods from the state of the art. Use of the new method results in a different product then the ones described in the state of the art. This can be shown by clear differences with regard to the properties of the monomers, e.g. according to FIG. 1 of WO 01/07055 and the mulitmers according to the invention. The invented molecules are surprisingly more appropriate for immune stimulation than the ones of the state of the art.

The molecules according to the invention can also be manufactured by providing 5′-phosphorylated oligodeoxyribonucleotide acids in water, if they are purified with an equivalent method to a polyacrylamide gel electrophoresis. Especially by the combined purification with a HPLC followed by a FPLC. It is known fork a person skilled in the art, that by the combination of several high performance methods like HPLC or FPLC a grade of purification can be reached which is analogue to the grade of a PAGE-purification.

Surprisingly the chronology of the single steps of the method leads to a mulitmeric molecule, comprising stably catenated monomers and at least 48 nucleotides (2 monomers with 24 nucleotides). The formed catena of molecules does not comprise free 5′- or 3′ ends. The monomers forming via intermolecular catenation the molecule according to the invention are characterized by:
comprising a sequence of at least 8 sequential nucleotides, fanning under suitable conditions with another part of this monomer a double stranded stem,
between these reverse complementary parts are at least 4 nucleotides
within the single stranded part CG-motives are present, which are recognized by cellular structures
modified nucleotides can also be part of a single stranded area, which are covalently linked to fatty acids, sugars or amino acids.

A molecule according to the invention comprises at least two monomers and is formed during the above-mentioned synthesis. The monomers are forming intermolecular catena of two, three, four, five or more. This results in the formation of so called di-, tri-, tetra-, penta- or hexamers, so called oligomers as shown in FIG. 1 (picture of gel after separation).

A molecule according to the invention can be also defined as catenate. In a preferred embodiment it is intended, that the molecule according to the invention and the catenated molecule, wherein at least one loop is linked to another loop of a further molecule, assemble with each other, so that preferably at least one, especially preferred several catenated elements are present.

In a further preferred embodiment of the invention it is intended, that several molecules with a self-complementary part of the loop hybridise with each other and form by this a product according to the invention.

Depending on the used sequences, a catenated molecule can be provided for instance, with several monomeric structures been preferably assembled to a catena by their respective loops.

A further multimeric structure is the one of the G-quartet. Guanine is able to form due to the orientation of its four H-bridge-binding-sides via guanine-guanine-base pairs a cyclic base quartet with eight H-bridges. A DNA-sequence, comprising several sequential guanosine nucleotides, is able to form a higher molecular helical structure, with the guanine bases showing a strong planarity with special staple interaction. Depending on the position, number and distribution of guanosine nucleotides within the sequence several G-structures may be formed.

A molecule according to the invention is able to modulate the activity of the human or animal immune system better, especially to activate, as molecules from the state of the art. The molecules from the state of the art are the known immune stimulatory nucleic acid sequences, operating as monomeric dumbbell-shaped structures. The most known immune modifying short oligodeoxyribonucleotide acid sequences comprise an unmethylated cytosine-guanosine-motive. A physiological effect of such nucleic acids is also understood as immune modulation respectively modulation of the activity of the immune system within the sense of the invention. The EP 1 196 178 for instance discloses several molecules, consisting of a stem with at least one loop, as they are disclosed for example in the FIGS. 1, 2 and 3. Within the sense of the invention such molecules are monomer structures. The present invention does not comprise such monomers as single molecules. It has to be noted that the term oligomer is used with several different meanings in science. An oligomer may be for instance a longer nucleic acid sequence as well as a structure comprising several of the same or similar molecules formed to a larger assembly. An oligomer within the sense of the invention designates catena of molecules, comprising at least 2-5 monomers forming so called dimers to pentamers. This relates to molecular weights according to FIG. 1 up to 200 kDa. Multimers within the sense of the invention would be for instance several stem-loop-structures according to EP 1 196 178, representing an assembly of several of the same or similar stem-loop-structures to a higher structure (a multimer). As multimers are all molecules according to the invention designated which are larger than 200 kDa. The described conditions for the reaction cause during the ligation a stabile intermolecular catenation of the ligations products. A resulting oligomer/multimer will be formed during the synthesis with respect to its confirmation only under the special reaction conditions. It is not possible to manufacture the mulitmers from monomers that have already been formed. The monomer structures forming the multimer are not covalently linked to each other, but they are linked as catenate structures. A formed oligomer (a person skilled in the art realises, that the feature of an oligomer in connection with a multimerization is not the meaning of oligodeoxyribonucleic acid sequence) or a multimer is stabile with respect to heat or denaturing agents, which means that the single molecule structures can not be obtained with simple physical means out of a molecule according to the invention.

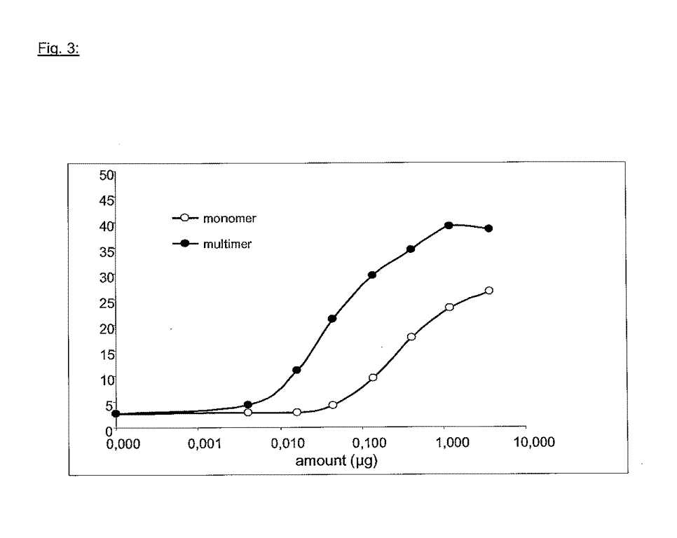

It is surprising, that such oligo- and mulitmeric structures have improved and not obvious properties compared to monomeric structures and can be obtained by comparatively simple method steps. The production of assemblies can for instance be performed via centrifugation, gel electrophoresis or column chromatography to show high complex structures, like for instance dimers, pentamers or others, which have compared to single molecule structures improved properties with regard to the modulation of the immune system (compare FIGS. 3 and 4, 5). This results in different forms of immune stimulation in lab organisms or humans.

All oligodeoxyribonucleotides according to the following characterization are suitable for the method of multimerization. Preferred is a mulitmer respectively a catenate according to the invention which is characterized by a oligodeoxyribonucleotide sequence used in the method comprising the following sequences:

a)
(SEQ ID No. 1)
agctgtagcagcttcggggggtatcgttcttcgtgtcgttcttagctgct
a-cagctgcagctgtagcagcttcggggggtatcgttcttcgtgtcgttc
t-tagctgctacagctgc
or
b)
(SEQ ID No. 2)
ggggttaccaccttctatagaaaacgttcttcggggcgttcttcatcgg-
taacccataggggttaccaccttctatagaaaacgttcttcggggcgttc
tt-catcggtaaccccta
or
c)
(SEQ ID No. 3)
ggggttaccaccttcattggaaaacgttcttcggggcgttcttaggtggt
aac-ccctaggggttaccaccttcattggaaaacgttcttcggggcgtt
cttaggtgg-taaccccta
or
d)
(SEQ ID No. 4)
agggttaccaccttcattggaaaacgttcttcggggcgttcttaggtgg-
taaccctcaggggttaccaccttcattggaaaacgttcttcggggcgt-
tcttaggtggtaacccctg
or
e)
(SEQ ID No. 5)
ggggttaccaccttcattggaaaacgttcttcggggcgttcttaggtgg-
taaccccggcgggttaccaccttcattggaaaacgttcttcggggcgttc
t-taggtggtaacccgcc
or
f)
(SEQ ID No. 6)
agggttaccacctteattggaaaacgttcttcggggcgttcttaggtggt
aaccc-taatgggttaccaccttcattggaaaacgttcttcggggcgttc
ttaggtgg-taacccatt
or
g)
(SEQ ID No. 7)
agggttaccaccttcattggaaaacgttcttcggggcgttcttaggtgg-
taaccctcaggggttaccacctteattggaaaacgttcttcggggcgttc
t-taggtggtaacccctg
or
h)
(SEQ ID No. 8)
CTAGGGGTTACCACCTACAAAAAAAAACGAAATTCGGGGCGAAGG-
GAGGTGGTAACCC
or
i)
and wherein the oligo deoxyribonucleotide sequence
has a length starting from 20 to 400 nucleotides.

The selection of the preferred sequences leads to molecules, which can be used surprisingly well for the stimulation of the immune system. It is especially preferred, whether the base sequence according to feature c) is comprised in the sequence ggggttac-caccttcattggaaaacgttcttcggggcgtt cttaggtggtaacccctaggggt-taccaccttcattggaaaacgttcttcggggcgttcttaggtggtaaccccta (SEQ ID No. 2) resp. according to d) agggttaccaccttcattggaaaacgttcttcgggg-cgttcttaggtggtaaccctcaggggttaccaccttcattggaa-aacgttcttcggggcgttcttaggtggtaacccctg (SEQ ID No. 4). Surprisingly the presence of such sequences of the single molecules results in an improved formation of catenate-like assembled molecules according to the invention.

In a further preferred embodiment of the invention it is intended, that the molecule comprises a partly single stranded, covalently closed chain of the deoxyribonucleotides. Within the catenated, oligomeric respectively multimeric structure of the molecule a partly single stranded covalently closed chain of deoxyribonucleotides is responsible for a long term effect of the molecule in the organism in which it is introduced.

In a further preferred embodiment of the invention it is intended, that the molecule comprises the base sequence N¹N²CGN³N⁴, wherein N¹N² is an element of the group of GT, GG, GA, AT or AA, N³N⁴ is an element of the group CT or TT, as well as C deoxycytosine, G deoxyguanosine, A deoxyadenosine and T deoxythymidine.

In an especially preferred embodiment it is intended, that the base sequence N¹N²CGN³N⁴ is positioned within the single stranded part of the closed chain of deoxyribonucleotides. Especially these preferred molecules show very strong effects during stimulation of the immune systems.

The molecule according to the invention thereby comprises not exclusively one deoxyribonucleotide molecule, wherein the deoxyribonucleotide acid molecule,
comprises a partly single stranded, dumbbell-shaped, covalently closed chain of deoxyribonucleotides and
comprises one or more sequences of a base sequence N¹N²CGN³N⁴
wherein N′N² is an element of the group of GT, GG, GA, AT or AA, N³N⁴ is an element of the group CT or TT, as well as C deoxycytosine, G deoxyguanosine, A deoxyadenosine and T deoxythymidine,
   characterized in, that the sequence comprises

a)
(SEQ ID No. 9)
GTTCCTGGAG ACGTTCTTAG GAACGTTCTC CTTGACGTTG GAGA-
GAAC
or
b)
(SEQ ID No. 10)
ACCTTCCTTG TACTAACGTT GCCTCAAGGAAGGTTGATCTT-
CATAACGTTGCCTAGATCA is,
or
c)
(SEQ ID Nr. 11)
a deoxyribonucleic acid sequene of the base
sequence AACGTTCTTCGGGGCGTT.

A multimer according to the application may comprise said monomer.

It is a matter of course, that a molecule according to the invention may have one or more substitutes bound via covalent binding. Such substitutes may be e.g. peptides, proteins, saccharides, antigenic structures, lipids, DNA and/or RNA.

The invention relates besides the above mentioned steps of a method for the manufacture of a product also to a method for the manufacture of a molecule comprising the following steps:
providing a 5′-phosphorylated oligodeoxyribonucleotide acid sequence in water purified by polyacrylamide gel electrophoresis,
lyophilisation until a dry residue is received followed by resuspension in a buffer,
adding a T4-DNA-ligase, forming a reaction mixture and
incubation of the reaction mixture at 37° C. for at least 30 minutes, or
the oligodeoxyribonucleotide acid sequence is provided after precipitation or lyophilisation in the presence of magnesium chloride, the T4-DNA-ligase is added and the received reaction mixture is incubated for at least 10 minutes at 37° C. preferably for at least 30 minutes, wherein the oligo deoxyribonucleotide acid is purified ahead of the precipitation or lyophilisation by a HPLC followed by a FPLC.

The same results with regard to the manufacture of mulitmers can be obtained with the precipitation or lyophilisation in the presence of magnesium chloride, especially if the oligodeoxyribonucleotide acid has been purified with a polyacrylamide gel electrophoresis, or with a combination of HPLC and FPLC.

It was completely surprising, that a different molecular structure as known from the state of the art (WO 2007/131495) or WO 01/07055) can be obtained by the method. As the methods show only differences in some steps the more surprising is has been, that this relatively slight modifications resulted in the manufacture of different molecules. Structures obtained with the method known from the state of the art (WO 01/07055 or WO 2007/131495) show clear differences in their properties. The molecules differentiate clearly with regard to the immune stimulatory effect, but also in other characteristics, like for instance side effects. Besides the different steps of the methods the use of the preferred sequences leads to the formation of a very specific reaction product with specific and outstanding properties. The use of sequences according to the invention together with the above mentioned method steps results in advantageous multimers, showing advantageous properties with regard to ones from the state of the art.

A catenate according to the invention comprises preferably 1+x single components, preferred partly single stranded, dumbbell-shaped covalently closed chains of deoxyribonucleotides,

wherein the single components have a stem and a loop, wherein the stem has at least 8 deoxyribonucleotides and the loop at least 4 deoxyribonucleotides and the loop has 1 to 6 CG-motives and x is an element from the set of all natural numbers.

The invention relates also to a composition, which comprises at least a molecule according to the invention and a chemotherapeutic. It was surprising that the surprising efficient stimulation of the immune system by a molecule according to the invention could be further improved surprisingly by combining the remedy according to the invention with known chemotherapeutics and using the composition preferably for instance for the treatment of tumours. Although it was known for a person skilled in the art, that monomers according to WO 01/07055 have an immune stimulatory effect and it was further known that chemotherapeutics have an effect on tumours, it was completely surprising that the polymers respectively mulitmers being formed by monomers cause in combination with chemotherapeutics an effect, being beyond aggregation. The elements of the composition according to the invention functionally interact leading to a synergistic effect. The elements combined in a composition according to the invention have an effect on the same aim to treat pathogens, especially tumours. Each element does not contribute to isolated results within the composition according to the invention, but the interaction between the single elements leads to the surprising effect. A composition according to the invention may be provided as a kit, with a molecule according to the invention and the chemotherapeutics according to the state of the art being provided separately. Thus, in a preferred embodiment the at least two components of the kits may be applied simultaneously or time delayed. The application of a composition according to the invention may for instance activate the immune system so that a subsequent application of a chemotherapeutic may have a very good effect. It is a matter of course, that it is possible to apply at first the chemotherapeutic and subsequently with a time delay a molecule according to the invention into the human or animal organism. For defined tumours the simultaneous application of a molecule according to the invention and the chemotherapeutic is preferred.

In a preferred embodiment of the invention a chemotherapeutic is selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

In a preferred embodiment of the invention the alkylating agents are selected from the group comprising
nitrogen mustard derivatives, especially
cyclophosphamide,
ifosfamide,
trofosfamide,
melphalan and/or
chlorambucil
alkylsulfonate, especially
busulfan, and/or
treosulfan
nitrosourea, especially
carmustine,
lomustine,
nimustine
estramustine and/or
streptozotocin
procarbazine and dacarbazine,
temozolomide and/or
thiotepa.

The alkylating agents have a very good effect on tumours, inhibiting their growth.

In a preferred embodiment of the invention the platinum analoga are selected from a group comprising:
cisplatin,
carboplatin and/or
oxaliplatin.

In a further preferred embodiment of the invention it is intended, that the intercalating agents are selected from the group comprising:
anthracycline, especially
doxorubicine (adriamycin),
daunorabicine,
epirubicine and/or
idarubicin,
mitoxantron,
amsacrine and/or
doxifluridine.

In a further preferred embodiment of the invention it is intended, that the antibiotics are selected from the group comprising:
bleomycin,
actinomycin D (dactinomycine) and/or
mitomycine.

It can be furthermore intended in another preferred embodiment of the invention as an advantage, that the mitoses suppressers are to selected form the group comprising:
alkaloids of vinca rosea, especially,
vinorelbine,
vincristine (oncovine),
vinblastine and/or
vindesine.

In a further especially preferred embodiment of the invention the taxanes are selected from the group comprising:
paclitaxel and/or
docetaxel.

Further it can be preferred, that the toposimerase suppressors are selected from the group comprising:
topoisomerase-I-inhibitors, especially
camptothecin,
topotecan and/or
irinotecan and/or
topoisomerase-II-inhibitors, especially,
etoposide,
teniposide.

Further it is preferred that in a special embodiment of the invention the anit-metabolites are selected from the group comprising:
folic acid antagonist, especially
methotrexat,

pyrimidin analoga, especially
5-flouridacil,
capecitabin,
cytosine arabinoside (cytarabin) and/or
gemcitabin,
purin analoga, especially
6-thiogunaine,
pentostatine,
azathioprine,
6-mercaptopurine,
fludarabin and/or
cladribine.

The invention relates further to a kit, comprising the molecule according to the invention and the chemotherapeutic, if applicable together with information about how to combine the content of the kit. The invention relates also—as already described—to a pharmaceutical comprising the molecule according to the invention or the composition if applicable with a pharmaceutical compatible carrier.

The invention relates further to the use of the molecule, the composition or the pharmaceutical for the manufacture of a remedy for the modulation of a human or animal immune system or for the modulation of the activity of the mentioned immune system. Modulation of the human or animal immune system each influence on the immune system shall be understood, having the effect that the immune system inhibits tumours or cancer. The modulation of the activity of the immune system can synonymously be understood to this or be described for a person skilled in the art as the known activities of the immune system that are directed against tumours and being surprisingly increased in their activity by remedies according to the invention. The modulation is especially stimulation or an increase of effects of the immune system respectively the immune system itself. Thus a remedy according to the invention can be used in a preferred embodiment to stimulate the T-cell mediated immune response but also the T-cell independent immune response. This process may comprise in a preferred embodiment of the invention a proliferation of B-cells or B-cell activation.

In an especially preferred embodiment the modulation of the activity of the immune system results in stimulation with the effect that cytokines are secreted respectively secretion is enhanced. It may be especially preferred that the molecule according to the invention respectively the composition according to the invention are used as adjuvant in therapeutic or prophylactic vaccination. The remedy according to the invention may be used very efficiently for the treatment of cell growth disorders, wherein in a preferred embodiment the cell growth disorder is a tumour disease. Preferably the tumour disease is a disease selected from the group comprising tumours of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endo-trine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.

In the following the invention is illustrated by examples without being limited to those examples.

Examples for the manufacture of a molecule according to the invention:

a) Manufacture of the monomer:

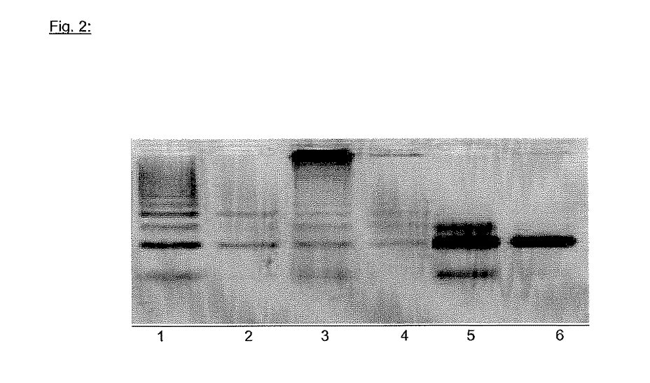

5′-phosphorylated oligodeoxyribonucleotide (ODN) with the sequence CCTAGGGGTTAC-CACCTTCATTGGAAAACGTTCTTCGGGGCGTTCTTAGGTGGTAACCCCTAGGGGT-TAC-CACCTTCATTGGAAAACGTTCTTCGGGGCGTTCTTAGGTGGTAACC (SEQ ID Nr. 12) were heated for 5 min to 90° C. and subsequently cooled on ice, to enable development of a hairpin structure. Self-complementary overhangs were ligated with a final concentration of 1 mg/ml DNA in the presence of T4-DNA Ligase (0.1 U/μg ODN) for 24 h at 37° C. Separation on a 1% agarose gel, each ligation product and after T7 digest, compare FIG. 2 lanes 5 and 6.

b) Manufacture of a molecule catena comprising di-, tri- and tetramers, so-called oligomers:

The oligodeoxyribonucleotide acid sequence CCTAGGGGTTACCACCTTCATTGGAA-AACGTTCTTCGGGGCGTTCTTAGGTGGTAACCCCTAGGGGTTACCACCTTCATTG-GAA-AACGTTCTTCGGGGCGTTCTTAGGTGGTAACC (SEQ ID Nr. 13) with a concentration of 1 mg/ml was precipitated with 0.3M sodium-acetate (pH 5.25), 10 mM MgCl₂ and a threefold volume of ethanol abs. After centrifugation (4° C., 13000 rpm) and washing of the pellet for one time with 70% EtOH, the ODN was dried at 50° C. for 10 min. Subsequently the resuspension in water was carried out (f.c.: 1 mg/ml). The ligation in the presence of T4-Ligase 2.3 U/μg ODN was done for 60 min at 37° C. Separation in 1% agarose gel, each ligation product and after T7 digest, compare FIG. 2, lanes 1 and 2.

c) Manufacture of a molecule catena comprising hexamers and multimers:

The oligodeoxyribonucleotide acid sequence CTAGGGGTTACCACCTACAAAAAAA-AACGAAATTCGGGGCGAAGGGAGGTGGTAACCC (SEQ ID Nr. 14) with a concentration of 1 mg/ml was precipitated with 0.3M sodium-acetate (pH 5.25), 10 mM MgCI₂ and a threefold volume of ethanol abs. After centrifugation (4° C., 13000 rpm) the ODN was dried at 50° C. for 10 min. The pellet was directly used for ligation (0.5 U/μg ODN) and incubated for 60 min at 37° C.

Separation in 1% agarose gel, each ligation product and after T7 digest, compare FIG. 2, lanes 3 and 4.

### Description of FIG. 2:

The ligation mixture, each first lane, respectively the product after T7 digest, each second lane, was applied to the lanes. A single band can be observed correlating to a single molecule, the monomer (lane 6 after T7 digest). Due to the lower molecular mass the migration is faster and is clearly different with regard to oligomers which are manufactured according to manufacture method b) and which are applied to lanes 1 and 2. Several bands can be observed above the band of the monomer representing di- to pentamers. In comparison to this the products manufactured according to method c) are larger and show clearly shorter way of migration, corresponding with larger molecules, compare lanes 3 and 4.

### Description of FIG. 1:

In order to determine the molecular weight the produced molecules were separated on a 3% agarose gel. On the left side next to the gel picture the molecular sizes of double-stranded DNA is shown and related to the respective migration distance. On the right next to the gel picture the high molecular products are designated. Lanes 1 to 3 were loaded with monomer manufactured according to a). Lane 3 shows the starting ODN. One observes a higher molecular structure, which is destroyed by heating at the beginning of the manufacture process. After ligation (lane 2) and T7 digest (lane 1) an enlarged molecules with regard to the starting ODN with a molecular mass of about 50 kDa can be observed corresponding to a monomer. It is clearly visible that no multimeric molecules are formed, in other words they will not be obtained during manufacture of monomers per se. Lane 4 shows again the starting ODN. In lane 5 the ligation mixture and lane 6 the ligation after T7 digest. The manufacture is performed according to the conditions as described in b) and c) for the manufacture of oligo- and multimeric molecules. It is clearly visible, that besides single molecules also the desired molecules according to the invention are formed. A band at about 100 kDa can be observed corresponding to a dimer, namely two catenated monomer molecules. The same is observed for tri- and tetramers.

Functional demonstration of molecules according to the invention:

Different cell culture experiments were done in order to prove the immune stimulatory properties of the molecules according to the invention. The ability to stimulate TLR9 was investigated by use of the murine macrophages of the cell line RAW 264. The cells were seeded with 125000 cells/cm² and after 16 h the monomeric (as control) and the oligomeric (according to b) an multimeric (according to c) molecules according to the invention were applied. After 7 h of incubation (37° C., 5% CO2) the cells were harvested and measured by fluorescence activated cell sorting (FACS). The results were used to generate a concentration-effect-curve, shown in FIG. 3.

The potency of the molecules according to the invention is increased by a factor of 10 (upper curve) in comparison to the monomeric single molecules (lower curve). Molecules according to the invention have a clearly better effect with less amounts used. The higher potency for immune stimulation can be attributed to a locally higher concentration achieved by the multimeric molecules which can especially in vivo not be achieved by higher doses, e.g. for reasons of the applicable amount.

### Stimulation of PBMCs for Cytokine Production

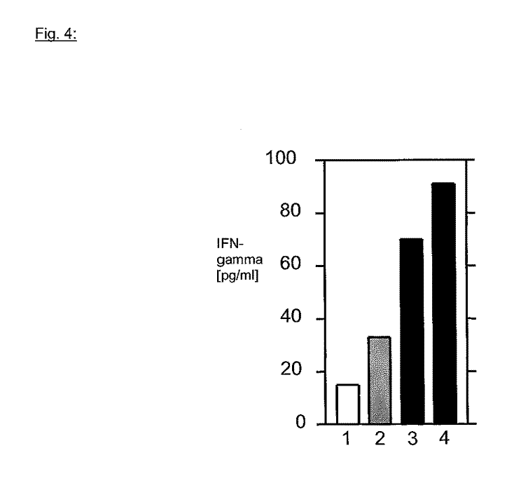

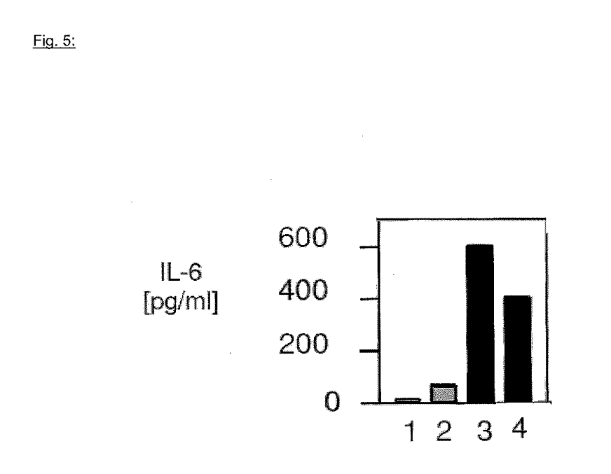

In order to perform stimulation assays peripheral mononuclear blood cells (PBMC) were isolated from whole blood or so-called “buffy coat”. The isolated cells (PBMC) were seeded in multiwell-plates. The first mixture contained not stimulated cells as negative control, the second mixture was stimulated as comparison to the monomers, the third with the oligomeric molecules and the fourth with the multimeric molecules. The secretion of the cytokines interferon-x, and interleukin 6 was determined by ELISA from the cell culture supernatant, compare FIGS. 4 and 5. According to FIG. 4 the stimulation of PBMCs with the oligo- and multimeric molecules results in a doubling of the INF-gamma secretion in comparison to monomeric single molecules. In which the multimeric molecules have a stronger effect in stimulation in comparison to the oligomeric molecules. FIG. 5 shows the IL-6 secretion due to stimulation. While the monomers show stimulation potential comparable to FIG. 4, the one of the molecules according to the invention is a multiple higher.

Molecules according to the invention are provided with the following sequences:

a)
(SEQ ID No. 1)
agctgtagcagcttcggggggtatcgttcttcgtgtcgttcttagctgct
a-cagctgcagctgtagcagcttcggggggtatcgttct-tcgtgtcgtt
ct-tagctgctacagctgc
or
b)
(SEQ ID No. 2)
ggggttaccaccttctatagaaaacgttcttcggggcgttcttcatcgg-
taacccataggggttaccaccttctatagaaaacgttct-tcggggcgtt
ctt-catcggtaaccccta
or
c)
(SEQ ID No. 3)
ggggttaccaccttcattggaaaacgttcttcggggcgttcttaggtgg-
taacccctaggggttaccaccttcattggaaaacgttct-tcggggcgtt
ct-taggtggtaaccccta
or
d)
(SEQ ID No. 4)
agggttaccaccttcattggaaaacgttcttcggggcgttcttaggtgg-
taaccctcaggggttaccaccttcattggaaaacgttct-tcggggcgt
tcttaggtggtaacccctg
or
e)
(SEQ ID No. 5)
ggggttaccaccttcattggaaaacgttcttcggggcgttcttaggtgg-
taaccccggcgggttaccaccttcattggaaaacgttct-tcggggcgtt
ct-taggtggtaacccgcc
or
f)
(SEQ ID No. 6)
agggttaccaccttcattggaaaacgttcttcggggcgttcttaggtggt
aaccc-taatgggttaccaccttcattggaaaacgttcttcg-gggcgtt
cttaggtgg-taacccatt
or
g)
(SEQ ID No. 7)
agggttaccaccttcattggaaaacgttcttcggggcgttcttaggtgg-
taaccctcaggggttaccaccttcattggaaaacgttcttcggggcgttc
ttaggtggtaacccctg oder
h)
(SEQ ID No. 8)
CTAGGGGTTACCACCTACAAAAAAAAACGAAATTCGGGGCGAAGG-
GAGGTGGTAACCC
or
i)
and wherein the oligodeoxyribonucleotide sequence
has a length from 20 to 400 nucleotides.

The nucleic acid sequences are not heated ahead of ligation and have a purification grade comparable to polyacrylamide electrophoresis. It can be provided by itself or by purification via HPLC followed by FPLC. The combination of HPLC and FPLC results in an equivalent purification grade to polyacrylamide electrophoresis. Subsequently the sequences are lyophilised until a dry residue is obtained. A resuspension in a buffer is then made and T4-DNA ligase is added followed from an incubation at 37° C. for 40 minutes. It was surprising, that the obtained concatenates cause an improved immune stimulation in mice. Surprisingly the combination of the of the concatenates according to the invention with chemotherapeutics results in an improved effect. The improved effect is surprisingly higher then the one of the single components and is beyond an additive effect. As chemotherapeutic antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines may be used.

1. Catenated molecule for the modulation of the activity of the human and animal immune system which is manufactured by a method comprising the following steps:
providing a 5′-phosphorylated oligonucleotide
alcohol precipitation or lyophilisation in the presence of MgCl₂, until a dry residue is obtained, followed by resuspension in a buffer
adding T4-DNA-ligase, thereby producing a reaction mixture, and
incubation of the reaction mixture at 37° C. for at least 30 minutes.

2. Molecule according to claim 1, characterised in that the catenated molecule is a molecule with at least one loop element being linked to another loop element of a second molecule, especially being interleaved linked, so that preferably at least one catenated element is present.

3. Molecule according to claim 1, characterized in that the oligodeoxyribonucleotide sequence comprises the following sequences:
a)
agctgtagcagcttcggggggtatcgttcttcgtgtcgttcttagctgct
acagctgcagctgtagcagcttcggggggtatcgttcttcgtgtcgttct
tagctgctacagctgc,
or
b)
ggggttaccaccttctatagaaaacgttcttcggggcgttcttcatcggt
aacccataggggttaccaccttctatagaaaacgttcttcggggcgttct
tcatcggtaaccccta,
or
c)
ggggttaccaccttcattggaaaacgttcttcggggcgttcttaggtggt
aacccctaggggttaccaccttcattggaaaacgttcttcggggcgttct
taggtggtaaccccta,
or
d)
agggttaccaccttcattggaaaacgttcttcggggcgttcttaggtggt
aaccctcaggggttaccaccttcattggaaaacgttcttcggggcgttct
taggtggtaacccctg,
or
e)
ggggttaccaccttcattggaaaacgttcttcggggcgttcttaggtggt
aaccccggcgggttaccaccttcattggaaaacgttcttcggggcgttct
taggtggtaacccgcc,
or
f)
agggttaccaccttcattggaaaacgttcttcggggcgttcttaggtggt
aaccctaatgggttaccaccttcattggaaaacgttcttcggggcgttct
taggtggtaacccatt,
or
g)
agggttaccaccttcattggaaaacgttcttcggggcgttcttaggtggt
taaccctcaggggttaccaccttcattggaaaacgttcttcggggcgttc
ttaggtggtaacccctg,
or
h)
ctaggggttaccacctacaaaaaaaaacgaaattcggggcgaagggaggt
ggtaaccc
and wherein
i)
the oligodeoxyribonucleotide sequence has a length
from 20 to 400 nucleotides.

4. Composition comprising a molecule according to claim 1 and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

5. Composition according to claim 4 characterized in that the alkylating agent is selected from the group comprising:
nitrogen mustard derivatives, especially
cyclophosphamide,
ifosfamide,
trofosfamide,
melphalan and/or
chlorambucil
alkylsulfonate, especially
busulfan, and/or
treosulfan
nitrosourea, especially
carmustine,
lomustine,
nimustine
estramustine and/or
streptozotocin
procarbazine and dacarbazine,
temozolomide and/or
thiotepa.

6. Composition according to claim 4, characterized in that the platinum analoga are selected from a group comprising:
cisplatin,
carboplatin and/or
oxaliplatin.

7. Composition according to claim 4, characterized in that the intercalating agents are selected from the group comprising:
anthracycline, especially
doxorubicine (adriamycin),
daunorubicine,
epirubicine and/or
idarubicine,
mitoxantron,
amsacrine and/or
doxifluridine.

8. Composition according to claim 4, characterized in that the antibiotics are selected from the group comprising:
bleomycine,
actinomycine D (dactinomycine) and/or
mitomycine.

9. Composition according to claim 4, characterized in that the mitoses suppressers are selected from the group comprising:
alkaloids of vinca rosea, especially,
vinorelbine,
vincristine (oncovine),
vinblastine and/or
vindesine.

10. Composition according to claim 4, characterized in that the taxanes are selected from the group comprising:
paclitaxel and/or
docetaxel.

11. Composition according to claim 4, characterized in that the toposimerase suppressors are selected from the group comprising:
topoisomerase-I-inhibitors, especially
camptothecin,
topotecan and/or
irinotecan and/or
topoisomerase-II-inhibitors, especially,
etoposide,
teniposide.

12. Composition according to claim 4, characterized in that the anti-metabolites are selected from the group comprising:
folic acid antagonist, especially
methotrexat,
pyrimidin analoga, especially
5-flouridacil,
capecitabin,
cytosine arabinoside (cytarabin) and/or
gemcitabin,
purin analoga, especially
6-thiogunaine,
pentostatine,
azathioprine,
6-mercaptopurine,
fludarabin and/or
cladribine.

13. Kit comprising a molecule according to claim 1 and/or a composition comprising a molecule according to claim 1 and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines and if applicable an information about combining the content of the kit.

14. Molecule according to claim 4 and the composition and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines for the use as medicament.

15. Pharmaceutical comprising a molecule according claim 1 and/or a composition comprising the molecule and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines if applicable together with a pharmaceutical compatible carrier.

16. Pharmaceutical according to claim 15, characterized in that the carrier is selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

17. Use of the molecule according to claim 1, the composition comprising the molecule and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines or the pharmaceutical comprising the molecule, for the manufacture of a remedy for the modulation of a human or animal immune system or for the modulation of the activity of the mentioned immune system.

18. Use according to claim 17, characterized in that the modulation is a stimulation or increase of the activity of the immune system.

19. Use according to claim 18, characterized in that the stimulation comprises a T-cell mediated or -independent immune response.

20. Use according to claim 19, characterized in that the immune response comprises a proliferation of B-cells and/or a B-cell activation.

21. Use according to claim 17, characterized in that the stimulation of the immune system comprises a secretion of cytokines.

22. Use according to claim 21, characterized in that the molecule and/or the composition is used as adjuvant in therapeutically or prophylactic vaccination.

23. Use of a molecule according to 22, the composition or the pharmaceutical for the manufacture of a remedy for the treatment of cell growth disorders.

24. Use according to claim 23, characterized in that the cell growth disorder is a tumour disease.

25. Use according to claim 24, characterized in that the tumour disease is a disease selected from the group comprising tumours of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicel-lular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.


SEQUENCE LISTING (14 sequences)

SEQ ID NO: 1 - DNA, 116 bp, Artificial Sequence
  Description of Artificial Sequence circular single-stranded with stem loop structure (dumbbell), all phosphodiester
agctgtagcagcttcggggggtatcgttcttcgtgtcgttcttagctgctacagctgcagctgtagcagcttcggggggtatcgttcttcgtgtcgttcttagctgctacagctgc

SEQ ID NO: 2 - DNA, 116 bp, Artificial Sequence
  Description of Artificial Sequence circular single-stranded with stem loop structure (dumbbell), all phosphodiester
ggggttaccaccttctatagaaaacgttcttcggggcgttcttcatcggtaacccctaggggttaccaccttctatagaaaacgttcttcggggcgttcttcatcggtaaccccta

SEQ ID NO: 3 - DNA, 116 bp, Artificial Sequence
  Description of Artificial Sequence circular single-stranded with stem loop structure (dumbbell), all phosphodiester
ggggttaccaccttcattggaaaacgttcttcggggcgttcttaggtggtaacccctaggggttaccaccttcattggaaaacgttcttcggggcgttcttaggtggtaaccccta

SEQ ID NO: 4 - DNA, 116 bp, Artificial Sequence
  Description of Artificial Sequence circular single-stranded with stem loop structure (dumbbell), all phosphodiester
agggttaccaccttcattggaaaacgttcttcggggcgttcttaggtggtaaccctcaggggttaccaccttcattggaaaacgttcttcggggcgttcttaggtggtaacccctg

SEQ ID NO: 5 - DNA, 116 bp, Artificial Sequence
  Description of Artificial Sequence circular single-stranded with stem loop structure (dumbbell), all phosphodiester
ggggttaccaccttcattggaaaacgttcttcggggcgttcttaggtggtaaccccggcgggttaccaccttcattggaaaacgttcttcggggcgttcttaggtggtaacccgcc

SEQ ID NO: 6 - DNA, 116 bp, Artificial Sequence
  Description of Artificial Sequence circular single-stranded with stem loop structure (dumbbell), all phosphodiester
agggttaccaccttcattggaaaacgttcttcggggcgttcttaggtggtaaccctaatgggttaccaccttcattggaaaacgttcttcggggcgttcttaggtggtaacccatt

SEQ ID NO: 7 - DNA, 116 bp, Artificial Sequence; identical to SEQ ID NO: 4
  Description of Artificial Sequence circular single-stranded with stem loop structure (dumbbell), all phosphodiester

SEQ ID NO: 8 - DNA, 58 bp, Artificial Sequence
  Description of Artificial Sequence circular single-stranded with stem loop structure (dumbbell), all phosphodiester
ctaggggttaccacctacaaaaaaaaacgaaattcggggcgaagggaggtggtaaccc

SEQ ID NO: 9 - DNA, 48 bp, Artificial Sequence
  Description of Artificial Sequence circular single-stranded with stem loop structure (dumbbell), all phosphodiester
gttcctggagacgttcttaggaacgttctccttgacgttggagagaac

SEQ ID NO: 10 - DNA, 60 bp, Artificial Sequence
  Description of Artificial Sequence circular single-stranded with stem loop structure (dumbbell), all phosphodiester
accttccttgtactaacgttgcctcaaggaaggttgatcttcataacgttgcctagatca

SEQ ID NO: 11 - DNA, 18 bp, Artificial Sequence
  Description of Artificial Sequence circular single-stranded with stem loop structure (dumbbell), all phosphodiester
aacgttcttcggggcgtt

SEQ ID NO: 12 - DNA, 116 bp, Artificial Sequence
  Description of Artificial Sequence circular single-stranded with stem loop structure (dumbbell), all phosphodiester
cctaggggttaccaccttcattggaaaacgttcttcggggcgttcttaggtggtaacccctaggggttaccaccttcattggaaaacgttcttcggggcgttcttaggtggtaacc

SEQ ID NO: 13 - DNA, 116 bp, Artificial Sequence; identical to SEQ ID NO: 12
  Description of Artificial Sequence circular single-stranded with stem loop structure (dumbbell), all phosphodiester

SEQ ID NO: 14 - DNA, 58 bp, Artificial Sequence; identical to SEQ ID NO: 8
  Description of Artificial Sequence circular single-stranded with stem loop structure (dumbbell), all phosphodiester